(19) United States
(12) Patent Application Publication
(10) Pub. No.: US 2007/0178058 A1
(43) Pub. Date: Aug. 2, 2007

(54) Methods of using stable ascorbic acid compositions

(76) Inventors: Jose E. Ramirez, Trumbull, CT (US); Joseph Faryniarz, Middlebury, CT (US)

Correspondence Address: CARTER, DELUCA, FARRELL & SCHMIDT, LLP, 445 BROAD HOLLOW ROAD, SUITE 225, MELVILLE, NY, 11747, US

(21) Appl. No.: 11/644,206

(22) Filed: Dec. 22, 2006

Related U.S. Application Data
(60) Provisional application No. 60/764,503, filed on Feb. 2, 2006.

(51) Int. Cl. A61K 8/41; A61K 8/49

(52) U.S. Cl. 424/70.28; 514/474

(57) ABSTRACT

Excellent percutaneous absorption of vitamin C is achieved by pre-treating skin with a cationic constituent such as cationic salt or solution thereof prior to application of a vitamin C composition.

### CROSS REFERENCE TO RELATED APPLICATION

This Application claims priority benefit of U.S. Provisional Application No. 60/764,503 filed Feb. 2, 2006 the entire disclosure of which is incorporated herein by this reference.

### BACKGROUND

1. Technical Field

The present disclosure relates to methods for attaining excellent percutaneous absorption of actives, such as vitamin C. More specifically, this disclosure relates to pre-treatment of an area of a user's skin with a cationic constituent to enhance penetration of an active.

2. Background Of Related Art

L-ascorbic acid is a water-soluble, antioxidant vitamin used in many products. For example, L-ascorbic acid is used in cosmetic, pharmaceutical and consumer products as an active ingredient for therapeutic treatment. L-ascorbic acid has therapeutic and corrective significance in that it is important in forming collagen, cartilage, muscle, and blood vessels. L-ascorbic acid also aids in the absorption of iron, and helps maintain capillaries, bones, and teeth. L-ascorbic acid further promotes healthy cell development, proper calcium absorption, normal tissue growth and repair. Moreover, L-ascorbic acid prevents blood clotting and bruising, while strengthening the walls of the capillaries. L-ascorbic acid is also important for healthy gums, protecting against infection, and assisting with the clearing up of infections, in reduction of cholesterol levels, high blood pressure and preventing arteriosclerosis. Consequently, deficiencies of ascorbic acid leads to problems such as scurvy, hemorrhages under the skin, bruising, poor wound healing, soft and spongy bleeding gums, loose teeth, edema, weakness, lack of energy, poor digestion, painful joints, bronchial infection and colds.

L-ascorbic acid is chemically defined as an alpha-ketolactone with the following chemical structure:

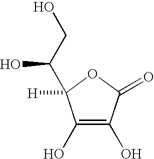

The number 2 and 3 carbons are double-bonded and contain acid-ionizable hydrogen in water (pK=4.2). Furthermore, ascorbic acid is moderately strong reducing agent. Unfortunately, these properties lead to instability in the ascorbic acid structure which is burdensome to formulators attempting to prepare ascorbic acid solutions. In particular, at higher pH's, the ascorbic acid increasingly becomes the unstable ascorbate anion (the conjugate base of ascorbic acid), which is susceptible to degradation.

The instability of ascorbic acid may be caused by a number of factors including stereochemical strain. For example, when the 2-hydroxy group ionizes, it places two negative charges in close proximity which favors ring disruption. Furthermore, oxidative degeneration likely promotes instability due to the ascorbate anion's propensity to act as a reductant, thus the molecule is prone to breaking down to form species such as L-threonic acid and oxalic acid. Such breakdowns can be catalyzed by the presence of a transition metal. Degradation may also occur due to a bulk water attack. Thus at lower ascorbate concentrations or ionic strength, water can react with ascorbic acid and degrade the molecule.

Various attempts have been made to produce stable solutions of L-ascorbic acid and its salts, but have been met with poor success. For example, U.S. Pat. No. 2,187,467 (the entire disclosure of which is incorporated herein by this reference) discloses aqueous solutions of ascorbic acid stabilized by the addition of salts of alkaline earth metals, ammonium, and soluble salt of a sulfite acid. However, this patent states that the stabilization was not achieved with the acid itself.

Other attempts at obtaining stable ascorbic acid compositions have been obtained by using expensive reagents and have also yielded a product with less desirable properties than ascorbic acid in its unmodified form.

The instability of L-ascorbic acid leads to a variety of disadvantages, including short shelf lives, required expiration dating, higher product costs, special storage considerations, product returns as well as reduced efficacy due to loss of active. Accordingly, compositions in which ascorbic acid is stable are desirable.

### SUMMARY

Compositions containing vitamin C and a reducing sugar exhibit excellent stability. Such compositions can be formulated using a reducing sugar and lead to products with satisfactory shelf life. The excellent stability also leads to product forms that were previously not obtainable, such as, for example, aqueous vitamin C, and solutions where vitamin C remains stable at pH's between 2-5. Furthermore, applying stable compositions of vitamin C having a pH below 5 increases the percutaneous absorption of vitamin C in skin. Moreover, pre-treating skin by topically applying a cationic constituent such as cationic salt or solution thereof, and then topically applying an aqueous solution of vitamin C has been found to increase the percutaneous absorption of vitamin C in skin.

### DESCRIPTION OF THE PREFERRED EMBODIMENTS

Stable vitamin C compositions for skin care in accordance with this disclosure are formulated in a manner which enables the vitamin C to remain stable when mixed with water. Compositions in accordance with some embodiments of this disclosure are effective in enhancing the penetration of vitamin C in the skin.

The compositions of the present disclosure contain vitamin C and a unique mixture of ingredients in an aqueous solution. The term “vitamin C” as used herein applies to substances that possess antiscorbutic activity. Such substances include, for example, L-ascorbic acid, commonly called ascorbic acid, salts of L-ascorbic acid, L-dehydroascorbic acid and salts of L-dehydroasorbic acid. L-ascorbic acid is a well known compound of general formula:

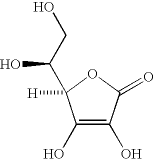

Suitable salt forms of vitamin C include any salt formed from the neutralization of ascorbic acid. Illustrative examples include sodium ascorbate formed by the neutralization ascorbic acid with sodium to form L-ascorbic acid-monosodium salt. Other useful forms include calcium ascorbate, magnesium ascorbate, potassium ascorbate, manganese ascorbate, zinc ascorbate, molybdenum ascorbate, chromium ascorbate, and combinations thereof.

The vitamin C is present in amounts that provide a benefit to the skin of a user. In embodiments, vitamin C is present in an amount sufficient to promote therapeutic or corrective treatment of a user's skin. The vitamin C present may be in acidic form, salt form, or mixtures thereof. As an illustrative example, amounts of vitamin C between about 5 to about 40% by weight of the total composition may be suitable. In embodiments, vitamin C is present in an amount between about 15 to about 25% by weight of the total composition. In other embodiments, the amount of vitamin C is between about 18 to about 22% by weight of the total composition.

The aqueous solution can further include water, one or more reducing sugars, one or more antimicrobial preservatives, one or more salts, one or more reducing agents, one or more surfactants, one or more conditioners such as Na Hyalurate, fragrance, and combinations thereof. In embodiments, purified water is used, such as, for example de-ionized water or USP water.

Suitable reducing sugars include sugars with a ketone or aldehyde group such that the sugar is capable of acting as a reducing agent. Non-limiting examples of reducing sugars include mannitol, sorbitol, xylitol, maltitol, lactitol, or combinations thereof. In vitamin C and reducing sugar compositions, it is believed that the reducing sugar oxidizes first and delays the start of any oxidation of the vitamin C, so that excessive oxidation in water is delayed or totally avoided. In embodiments, the reducing sugars are present between about 0.1% w/v to about 10.0% w/v of the total formulation. In embodiments, the reducing sugars are present between about 0.5% w/v to about 5.0% w/v of the total formulation.

Optionally, the reducing sugars may be mixed with water to form a reducing sugar solution that can be used to formulate a stable vitamin C composition in accordance with this disclosure. The reducing sugar solution may contain, for example, reducing sugar in an amount between about 1% and about 99% by weight. In embodiments, the reducing sugar solution may contain about 70% by weight of the reducing sugar. The amount of reducing sugar solution used to formulate the stable vitamin C composition will depend upon a number of facts including the concentration of reducing sugar in the solution. Typically, however, for a 70% solution, the reducing sugar solution may be added to the composition to between about 0.25% v/v to about 10.0% v/v of the formulation. In embodiments, such reducing sugar solution is admixed between about 1% to about 5% v/v of the total formulation.

In embodiments in accordance with the present disclosure, sorbitol can be used as a reducing sugar. Sorbitol, also known as glucitol is a sugar alcohol having the general formula:

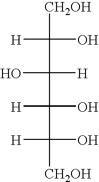

Sorbitol is a sugar alcohol (also known as polyol, polyhydric alcohol, or polyalcohol) which is a hydrogenated form of carbohydrate, whose carbonyl group (aldehyde or ketone, reducing sugar) has been reduced to a primary or secondary hydroxyl group. In one embodiment, sorbitol is mixed with water to form a 70% solution suitable for use as an ingredient in forming compositions in accordance with the present disclosure.

Antimicrobial preservatives can be used to prevent or inhibit the growth of micro-organism which could present a risk of infection to the user or degrade the vitamin C. Thus, suitable antimicrobial preservatives include ingredients capable of retarding the oxidation of vitamin C and/or extending the shelf-life of active ingredients. The properties of these antimicrobial substances typically include chemical groups that are aggressive towards living cells. Examples of suitable preservatives include quaternary ammonium salts, phenoxyethanol, amine salts, Na Metabisulfite and combinations thereof. The antimicrobial preservatives may be present between about 0.1% w/w to about 5.0% w/w of the formulation.

Suitable salts that may be employed in making stable vitamin C compositions in accordance with the present disclosure include acid and base addition salts. Non-limiting illustrative examples of such acid salts include: inorganic acid addition salts such as hydrochloride, sulfate, and phosphate; and organic acid addition salts such as acetate, maleate, fumarate, tartrate, and citrate. Examples of suitable basic salts include: metal salts such as the alkali metal salts such as the sodium salt and potassium salt; alkaline earth metal salts such as magnesium salt and calcium salt; and other salts such as aluminum salt, and zinc salt. Examples of suitable ammonium salts are ammonium salt and tetramethylammonium salt. Examples of suitable amine addition salts are salts with morpholine and piperidine. Examples of suitable amino acid addition salts include salts with lysine, glycine, and phenylalanine. The one or more salts may be present in a between about 0.01% w/w to about 4.0% w/w of the formulation. In embodiments mixtures of salts have been found to further promote stability, especially when combined with a reducing sugar such as sorbitol. In embodiments, Ca hydroxide (e.g., between about 0.01-0.5% w/v of the total formulation) is combined with Zn Chloride (e.g., between about 0.01-2.0% w/v of the total formulation). The combination of salt admixtures with reducing sugar was found to promote stability of aqueous ascorbyl acid solutions.

In embodiments, alkaline earth metal salts such as magnesium and calcium salts can be provided in a unique stability promoting admixture. It is believed that the combination of such metal ions in solution promotes the stability of the compositions. Other salt mixtures such as zinc salts and aluminum salts also promote stability.

In embodiments, the stable formulations can optionally include surfactants. Suitable surfactants for use with the compositions of the present disclosure include ionic or nonionic surfactants, used alone or in admixture. Illustrative surfactants include cetearyl alcohol and sodium cetearyl sulfate, alkyldimethylbenzylamines, PEG-1000 monocetyl ether, or quaternary ammonium salts such as alkyl trimethyl ammonium bromide; polyol ester glycerol monostearate and potassium stearate, sodium lauryl sulfate, ethoxylated fatty alcohols and combinations thereof. Fatty acids like stearic acids may be included to regulate the consistency of the composition. Optionally, polymers such as carbomers can be included in the present composition. Particularly useful surfactants for use in the aqueous phase are sodium lauryl sulfate, saponins or combinations thereof. The surfactants may be present between about 0.0 and about 20% by weight of the total composition. In embodiments, the surfactants are present in an amount between about 0.1 and about 5% by weight of the total composition.

It has been found that the inclusion of various surfactants in compositions of the present disclosure increases the percutaneous absorption of vitamin C when solutions are applied to skin. By adding surfactants to the stable ascorbic acid solutions, the surface tension of the solution is decreased allowing for better absorption through skin. Thus methods of applying surfactant containing solutions to skin in order to increase the levels of vitamin C absorbed into the skin are also described herein. Accordingly, surfactant may also be included in the compositions of the present disclosure in amounts sufficient to increase the absorption of vitamin C through skin. For example, by adding SLS (30% solution) in an amount of about 0.1 to 5% by weight of the total composition, and then applying the solution to skin, the percutaneous absorption of vitamin C through skin is increased in comparison to vitamin C solutions that do not have surfactant.

The pH of the aqueous solution can be adjusted to be between about 2 to about 6, and, in some particularly useful embodiments below 5. The pH of the composition ensures that most of the ascorbic acid remains in the protonated, uncharged form. The protonated form of ascorbic acid used in compositions of the present disclosure is believed to remove the ionic repulsion of the two oxygen groups, thus helping to stabilize the molecule. Also because the protonated form of ascorbic acid is uncharged, entry into the skin (which itself has a pH of about 3-5) is believed to be facilitated. Agents suitable for adjusting the pH of the aqueous phase include, but are not limited to citric acid, phosphoric acid, lactic acid or glycolic acid. The pH adjustment agents may be present between about 0.01 and about 5% by weight of the total composition. In embodiments, the pH adjustment agent is present in an amount of about 0.1 to about 1.0% by weight of the total composition.

Suitable reducing agents that can be used in the present compositions include, but are not limited to propyl gallate and sulfites, including but not limited to sulfites, bisulfites, metabisulfites, their salts, and their derivatives, in embodiments sodium metabisulfite. Since vitamin C has a tendency to oxidize, these antioxidants may be advantageous because they have greater tendencies to oxidize than vitamin C. Sodium metabisulfite has the added advantage that it does not discolor by oxidation. In vitamin C and sodium metabisulfite compositions, it is believed that the sodium metabisulfite oxidizes first and delays the start of any oxidation of the vitamin C, so that excessive oxidation is delayed or totally avoided. The reducing agents may be present between about 0.1 and about 10% by weight of the total composition. In some embodiments, the reducing agents are present in an amount of about 0.5 to about 5% by weight of the total composition.

The aqueous phase can be prepared by mixing the various ingredients while mixing and heating to 70-75° C.

Other suitable optional ingredients include moisturizing agents such as Na Hyalurate solution and fragrance. Na Hyalurate moisturizing agent that is synonymous with and refers to hyaluronic acid, sodium salt; sodium hyaluronate; hyaluronic acid; or sodium hyalurate and has the general formula (C₁₄H₂₀NO₁₁Na)ₙ. Na hyalurate 1% solution may be present, for example, in an amount of about 0.001 to about 0.2% w/v of the total composition, or in amounts the effectively moisturize the formulations.

The viscosity of the final vitamin C composition can be between about 30 to 10,000 centipoise (cps), in embodiments between about 30 and about 250 cps. The specific gravity of the final composition can be between about 1.0 and 1.15, in embodiments between about 1.02 and about 1.06.

The final vitamin C composition may be a substantially clear, viscous liquid to a semi-viscous lotion. Those skilled in the art will envision testing to confirm the shelf life of the products described herein. Further testing methodology is described below.

The present disclosure also relates to methods of pre-treating skin to increase the percutaneous absorption of vitamin C there through. It has been found that the application of cationic constituent such as cationic salt or cationic solution to skin prior to the application of vitamin C compositions of the present disclosure increases the percutaneous absorption of vitamin C into the skin. Cationic constituents such as salt or solutions thereof are applied to skin in amounts sufficient to increase the percutaneous absorption of vitamin C. For example, a small volume of a cationic salt-containing pretreatment solution (such as, for example, an aqueous 5% alkyldimethylbenzylamine solution) may be applied to skin prior to the application of a vitamin C solution. The skin is dried and the vitamin C solution is added to the skin.

In accordance with the present disclosure, one or more cationic constituents can be applied in amounts that provide the benefit to the skin of the user, such as in an amount sufficient to coat skin surface with positively charged molecules. As used herein, cationic constituent refers to any ingredient with one or more positively charged moieties that apply positive charge to skin. Non-limiting examples of cationic constituents include one or more cationic molecules such as cationic salts, positively charged amino acids, quaternary ammonium salts, cationic polymers, and combinations of these cationic constituents.

In embodiments, cationic salts suitable for use as a pre-treatment in accordance with the present disclosure include cationic salts that are not substantially toxic at the dosage administered to achieve the desired effect and do not independently possess significant pharmacological activity. Non-limiting examples of suitable cationic salts include cationic salts formed at any acidic (e.g., carboxyl) group; multivalent cationic salts, including cations of the alkaline earth metals (Group IIA), transition metals (Groups IIB, IVB, VB, VIIB, VIIB, VIIIB, IB, IIB, IIA, IVA) and non-metal elements (Groups IVA, VA) for use alone or combined together, or in combination with other cationic constituents described herein. In embodiments, cationic salts include positively charged salts of carboxymethylcellulose (CMC). In embodiments, cationic salts include the alkali metal salts (such as, for example, sodium and potassium), alkaline earth metal salts (such as, for example, magnesium and calcium), and organic salts.

Pharmaceutically acceptable cationic salts suitable for use in accordance with the present disclosure include any salts formed by neutralization of the free carboxylic acid group of pharmacologically active compounds. The neutralization may occur by contacting the carboxylic acid containing compounds with a base of a pharmaceutically acceptable metal, ammonia or amine. Non-limiting examples of such metals are sodium, potassium, calcium and magnesium. Non-limiting examples of such amines are N-methylglucamine and ethanolamine.

In embodiments, cationic salts include those organic primary, secondary and tertiary amines, as for example, trialkylamines, including triethylamine, procaine, dibenzylamine, 1-ethenamine, N,N′-dibenzylethylenediamine, dihydroabiethylamine, N-(lower)alkylpiperidine, and any other suitable amine. In embodiments, alkyldimethylbenzylamine salts are particularly useful cationic salts.

In embodiments, suitable cationic constituents contain amino acids having a positive charge. Non-limiting examples of suitable amino acids which can be positively charged include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In embodiments, basic amino acids are suitable for use herein. Basic amino acids are those amino acids where in a neutral solution, the R group of the amino acid can gain a proton and become positively charged. Non-limiting examples of suitable basic amino acids include lysine, arginine, and histidine.

Non-limiting examples of suitable quaternary compounds include: benzalkonium chlorides and/or substituted benzalkonium chlorides, dialkyl quaternary, N-(3-chloroallyl) hexaminium chlorides, benzethonium chloride, methylbenzethonium chloride, and cetylpyridinium chloride. In embodiments, benzalkonium chloride such as, for example, a 5% solution can be applied to the skin before application of an active solution such as a vitamin C formulation.

Non-limiting examples of cationic polymer suitable for use in accordance with the present disclosure include: cationic polysaccharides, cationic copolymers of saccharides and synthetic cationic monomers, and synthetic polymers.

Non-limiting examples of cationic polysaccharides includes polymers based on a 5 or 6 carbon sugars and derivatives, which have been made cationic by adding cationic moieties onto the polysaccharide backbone. Such cationic polysaccharides can be composed of one type of sugar or of more than one type, i.e. copolymers. The monomers may be in straight chain or branched chain geometric arrangements. Additional suitable cationic polysaccharide polymers include: cationic celluloses and hydroxyethyl celluloses, cationic starches and hydroxyalkyl starches, cationic polymers based on arabinose monomers, cationic polymers derived from xylose polymers, cationic polymers derived from fucose polymers, cationic polymers derived from fructose polymers, cationic polymers based on acid-containing sugars such as galacturonic acid and glucuronic acid, cationic polymers based on amine sugars such as galactosamine and glucosamine, cationic polymers based on 5 and 6 membered ring polyalcohols, cationic polymers based on galactose monomers which occur in plant gums and mucilages, cationic polymers based on mannose monomers, and cationic polymers based on galactomannan copolymer known as guar gum.

The cationic copolymers of saccharides and synthetic cationic monomers in accordance with the present disclosure include those containing the following saccharides: glucose, galactose, mannose, arabinose, xylose, fucose, fructose, glucosamine, galactosamine, glucuronic acid, galacturonic acid, and 5 or 6 membered rinse polyalcohols. Also included are hydroxymethyl, hydroxyethyl and hydroxypropyl derivatives of the above saccharides. When saccharides are bonded to each other in the copolymers, they may be bonded via any of several arrangements, such as 1,4-.alpha.; 1,4-.beta.; 1,3-.alpha.; 1,3-.beta. and 1,6 linkages. The synthetic cationic monomers for use in these copolymers can include dimethyidiallylammonium chloride, dimethylaminoethylmethyacrylate, diethyldiallyl ammonium chloride, N,N-diallyl,N-N-dialklyl ammonium halides, and the like.

Non-limiting examples of copolymers of saccharides and synthetic cationic monomers include those composed of cellulose derivatives (e.g. hydroxyethyl cellulose) and N,N-diallyl,N-N-dialkyl ammonium chloride.

Non-limiting examples of cationic synthetic polymers include: cationic polyalkylene imines, cationic ethoxypolyalkylene imines, cationic poly[N-[3-(dimethylammonio)propyl]-N′[3-(ethyleneoxyethylene dimethylammonio)propyl]urea dichloride], and polymers having a quaternary ammonium or substituted ammonium ion. In embodiments, cationic polymeric skin conditioning agents are those cationic polysaccharides of the cationic guar gum with molecular weights of 1,000 to 3,000,000. Such polymers have a polysaccharide backbone having galactomannan units and a degree of cationic substitution ranging from about 0.04 per anydroglucose unit to about 0.80 per anydroglucose unit with the substituent cationic group being the adduct of 2,3-epoxypropyl-trimethyl ammonium chloride to the natural polysaccharide backbone.

Additional non-limiting examples of cationic polymers include the polymerized materials such as certain quaternary ammonium salts, copolymers of various materials such as hydroxyethyl cellulose and dialkyldimethyl ammonium chloride, acrylamide and beta methacryloxyethyl trimethyl ammonium methosulfate, the quaternary ammonium salt of methyl and stearyl dimethylaminoethyl methacrylate quaternized with dimethyl sulfate, quaternary ammonium polymer formed by the reaction of diethyl sulfate, a copolymer of vinylpyrrolidone and dimethyl aminoethylmethacrylate, quaternized quars and guar gums and the like.

In embodiments, cationic polymers include: polyquaternium, -5(the copolymer of acrylamide and beta-methacrylyloxyethyl trimethyl ammonium methosulfate), -6(a polymer of dimethyl diallyl ammonium chloride), -7(the polymeric quaternary ammonium salt of acrylamide and dimethyl diallyl ammonium chloride monomers), and combinations of these cationic polymers.

In embodiments, cationic polymers include: hydroxypropyl guar gum, and/or guar hydroxypropyltrimonium chloride.

Quantities of such a cationic polymer are generally a minimum of about 0.05 wt. % of the formulation. Generally, the maximum quantity is no more than about about 10 wt. % of the formulation.

In accordance with the present disclosure, cleanser preparatory compositions can be applied to skin in amounts that provide the benefit to the skin of the user, such as in an amount sufficient to remove dirt and oil from the skin. Generally, the cleansers are soap-free and include water, detergent, surfactant, humectants, skin conditioning agent, PH adjustor, extracts, preservatives, fragrance and colorant, however, any cleanser suitable for removing dirt and oil from skin may be used. Suitable cleansers are commercially available and typically include a combination of anionic, cationic, amphoteric and/or non-ionic surfactants in an aqueous vehicle. The cleanser advantageously can include a combination of compounds to compensate for the well known fact that cleansing agents, by their very nature, are not always well tolerated by the skin. The oil-removal feature of a cleanser can result in drying of the skin, and skin irritation. By incorporating various protective agents in the cleanser process the cleanser overcomes shortcomings found in many alternative products. One commercially available preparatory composition is Obagi Nu-Derm® cleanser from OMP, Inc. of Long Beach, Calif. The Obagi Nu-Derm® cleanser contains a combination of water, cocamidopropyl betaine, sodium lauroyl oat amino acids, sodium laureth sulfate, glycerin, aloe barbadensis gel, glycerth-7, apricot triethanolamine, sage extract, borage extract, phenoxythanol, methylparaben, propylparaben, ethylparaben, butylparaben, saponins, fragrance, and colorant.

Optionally, foaming gel preparatory composition can be applied in amounts that provide the benefit to the skin of the user, such as in an amount sufficient to remove dirt, oil and/or impurities to clean skin and leave it more receptive to treatment. Generally, foaming gels include water, detergent, surfactant, humectants, skin conditioning agent, PH adjustor, extracts, preservatives, fragrance and colorant; (however any foaming gel may be applied that cleans the skin by removing dirt and/or oil). One commercially available composition is Obagi Nu-Derm® foaming gel from OMP, Inc. of Long Beach, Calif. The Obagi Nu-Derm® foaming gel contains a combination of water, sodium lauryl oat amino acids, cocamidopropyl betaine, sodium laureth sulfate, aloe barbadensis gel, alfalfa extract, borage extract, sodium chloride, xantham gum, saponins, phenoxythanol, methylparaben, propylparaben, ethylparaben, butylparaben, fragrance and colorant. This cleanser frees the skin of pollutants without damaging the skin's own natural moisture content.

Optionally, toner can be applied in amounts that provide the benefit to the skin of the user, such as in an amount sufficient to hydrate and tone skin while reducing the pH of the skin. Toner may also help by removing dirt, oils, and grime without overly drying out sensitive skin. Generally, toners include water, skin conditioner, astringent, minerals, moistening agent, vitamins and complexes thereof, anti-microbial, cleanser, extract, surfactant, anti-irritant, fragrance and colorant; however any commercially available skin toner may be used. One commercially available composition is Obagi Nu-Derm® toner available from OMP, Inc. of Long Beach, Calif. to ameliorate the potentially harsh or drying effects of witch hazel. The Obagi Nu-Derm® toner contains a combination of water, aloe barbadensis gel, witch hazel distillate, potassium alum, sodium PCA, panthenol, DMDM hydantion, polysorbate 80, allantoin, sage extract, calendula officinalis extract, saponins, fragrance, and colorant.

The following non-limiting examples further illustrate compositions and methods in accordance with this disclosure.

### EXAMPLES

Example 1 below shows suitable compositions in accordance with the present disclosure.

| Ingredient | % of solution (w/v) |
|---|---|
| Water | 60–96 |
| Sorbitol 70% | 0.1–10 |
| Ca Hydroxide | 0.10–0.5 |
| Zn Chloride | 0.10–2 |
| Na Hyalurate | 0.001–0.02 |
| Ascorbic acid | 5–40 |
| SLS (30% solution) | 0–5 |
| Phenoxyethanol | 0.1–1 |
| Fragrance | 0.0–5 |
| Alkydimethylbenzylamine | 0–5 |

Example 2 below shows other suitable compositions in accordance with the present disclosure.

| Ingredient | % of solution (w/v) |
|---|---|
| Water | 60–96 |
| Reducing Sugar | 0.1–10 |
| Metal Salt or mixtures thereof | 0.10–5 |
| Moisturizing Agent | 0.0–0.02 |
| Ascorbic acid | 5–40 |
| Antimicrobial | 0.0–1 |
| Surfactant | 0.0–5 |
| Fragrance | 0.0–5 |

The compositions of the present disclosure may be packaged in suitable containers such as tubes or bottles. Suitable containers are commercially available from a variety of suppliers. A wide variety of containers and suppliers are listed in the CPC Packaging Directory. (See, Buyers' Guide under “Containers” at www.cpcpkg.com). In embodiments, containers are selected with low oxygen permeability. Suitable containers include high density polyethylene and the like.

### Example 3

### Stability Study

In vitamin C compositions without reducing sugar, an aqueous solution of 5% ascorbic acid will likely decompose to less than 90% of the concentration at room temperature in 4 weeks time. See for example U.S. Pat. No. 4,983,382 the entire disclosure of which is incorporated herein by this reference.

Conversely, the stability of compositions made in accordance with the present disclosure show improved stability. Such compositions were evaluated by placing aliquots of each example in an oven at 5, 25, 30 and 40 degrees Centigrade for predetermined time periods and at the end of each time period analyzing the amount of vitamin C present in the composition.

The following results were observed with compositions in accordance with example 1 having 20% initial vitamin C concentration, sorbitol 70%, Ca hydroxide, Zn chloride, Na hyaluronate 1%, SLS (30% solution), phenoxyethanol and fragrance.

| Stability of formulation of | |
|---|---|
| example No. 1 | % Vitamin C |
| Initial amount of Vit. C | 20% |
| 6 weeks at 40 C.: | 16.52% |
| 3 months at 25 C.: | 17.50% |
| 3 months at 40 C.: | 13.23% |
| 8 months at 25 C. | 16% |

### Conversely, vitamin C composition without reducing sugar showed only 9.7% vitamin C remaining after 3 months at 40° C.

### Example 4

### Percutaneous Absorption Study:

The in-vitro percutaneous absorption of vitamin C formulations were compared using intact human cadaver skin. Cumulative transdermal absorption of radiolabeled [¹⁴C] L-ascorbic acid was measured at 24 hours. The human cadaver skin was obtained from a single donor and dermatomed to approximately 500 micron thickness. The skin samples were mounted on Franz static diffusion glass chambers. The skin surfaces of approximately 1.77 cm² were washed with 0.5 ml of water at 37° C. for 10 seconds. The water was aspirated and the surface pad dried. The following treatments were performed.
Treatment A. 15 mg of a formulation in accordance with example 1 having 25% ascorbic acid, reducing sugar, and metal ions was applied to 1.77 cm² of human cadaver skin samples.
Treatment B: Skin pretreated with 15 mg of pretreatment solution (5% benzalkonium chloride, a cationic salt). The skin was dried, and then 15 mg of formulation of Treatment A was applied to the skin.

TABLE A

AMOUNT OF
ASCORBIC ACID
ABSORBED DERMIS (24 hrs.)

| | | as % of amount |
|---|---|---|
| Treatment | In micrograms | applied |
| A | 26.5 | 8.0 |
| B | 93.1 | 33.6 |

Pre-treating the skin by topically applying a cationic salt or solution thereof to the skin enhanced absorption of vitamin C into the dermis, compared to skin that was not pretreated. Dermal levels were observed as increasing by approximately 4 times.

### Example 5

### Percutaneous Absorption Study:

The in-vitro percutaneous absorption of vitamin C formulations are compared using intact human cadaver skin. Cumulative transdermal absorption of radiolabeled [¹⁴C] L-ascorbic acid is measured. The human cadaver skin is obtained from a single donor and dermatomed to approximately 500 micron thickness. The skin samples are mounted on Franz static diffusion glass chambers. Skin surfaces of approximately 1.77 cm² are washed with 0.5 ml of water at 37° C. for 10 seconds. The water is aspirated and the surface pad dried. The following treatments are performed.
Treatment A. 15 mg of a formulation in accordance with example 1 having 25% ascorbic acid, reducing sugar, and metal ions is applied to 1.77 cm² of human cadaver skin samples.
Treatment B: Skin pretreated with 15 mg of pretreatment solution (5% polyquaternium, -5(the copolymer of acrylamide and beta-methacrylyloxyethyl trimethyl ammonium methosulfate, a cationic polymer)). The skin is dried, and then 15 mg of formulation of Treatment A is applied to the skin.

Percutaneous absorption of Vitamin C in Treatment B is expected to be greater than that of Treatment A.

### Example 6

### Percutaneous Absorption Study:

The in-vitro percutaneous absorption of vitamin C formulations are compared using intact human cadaver skin. Cumulative transdermal absorption of radiolabeled [¹⁴C] L-ascorbic acid is measured. The human cadaver skin is obtained from a single donor and dermatomed to approximately 500 micron thickness. The skin samples are mounted on Franz static diffusion glass chambers. Skin surfaces of approximately 1.77 cm² are washed with 0.5 ml of water at 37° C. for 10 seconds. The water is aspirated and the surface pad dried. The following treatments are performed.
Treatment A. 15 mg of a formulation in accordance with example 1 having 25% ascorbic acid, reducing sugar, and metal ions is applied to 1.77 cm² of human cadaver skin samples.
Treatment B: Skin is pretreated by cleansing with cleanser composition and toning with a toner composition. Skin is further pretreated with 15 mg of pretreatment solution (5% arginine, a basic amino acid). The skin is dried, and then 15 mg of formulation of Treatment A is applied to the skin.

Percutaneous absorption of Vitamin C in Treatment B is expected to be greater than that of Treatment A.

### Example 7

### Percutaneous Absorption Study:

The in-vitro percutaneous absorption of vitamin C formulations are compared using intact human cadaver skin. Cumulative transdermal absorption of radiolabeled [¹⁴C] L-ascorbic acid is measured. The human cadaver skin is obtained from a single donor and dermatomed to approximately 500 micron thickness. The skin samples are mounted on Franz static diffusion glass chambers. The skin surfaces of approximately 1.77 cm² are washed with 0.5 ml of water at 37° C. for 10 seconds. The water is aspirated and the surface pad dried. The following treatments are performed.
Treatment A. 15 mg of a formulation in accordance with example 1 having 25% ascorbic acid, reducing sugar, and metal ions is applied to 1.77 cm² of human cadaver skin samples.
Treatment B: Skin pretreated with cleanser, allowed to dry, then 15 mg of pretreatment solution (5% Guar hydroxypropyltrimonium chloride, a cationic polymer). The skin is dried, and then 15 mg of formulation of Treatment A is applied to the skin.

Percutaneous absorption of Vitamin C in Treatment B is expected to be greater than that of Treatment A.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

What is claimed is:

1. A method comprising:
pre-treating an area of a user's skin by topically applying a composition comprising a cationic constituent; and
topically applying a composition comprising Vitamin C to the pretreated area.

2. The method of claim 1 wherein the cationic constituent is selected from the group consisting of cationic salts, positively charged amino acids, quaternary ammonium salts, cationic polymers, and combinations thereof.

3. The method of claim 1 wherein the composition comprising a cationic constituent is an aqueous solution.

4. The method of claim 1 wherein the composition comprising a cationic constituent comprises cationic salt.

5. The method of claim 4 wherein the salt is selected from the group consisting of alkali metal salts, alkaline earth metal salts, light metal salts, organic primary salts, secondary and tertiary amines, and combinations thereof.

6. The method of claim 4 wherein the cationic salt is an alkyldimethylbenzylamine.

7. The method of claim 4 wherein the cationic salt is a benzalkonium chloride.

8. The method of claim 4 wherein the composition containing a cationic salt comprises a 5% aqueous solution of benzalkonium chloride.

9. A method comprising:
pre-treating an area of a user's skin by topically applying a composition comprising a cationic constituent selected from the group consisting of cationic salts, positively charged amino acids, quaternary ammonium salts, cationic polymers, and combinations thereof; and
topically applying a composition comprising a vitamin C composition to the pretreated area.

10. The method of claim 9 wherein the composition comprising a cationic constituent is an aqueous solution.

11. The method of claim 9 wherein the vitamin C composition is an aqueous solution.

12. The method according to claim 9 wherein percutaneous absorption of the Vitamin C is increased compared to application of the Vitamin C alone.
